(19) United States
(12) Patent Application Publication
(10) Pub. No.: US 2026/0315684 A1
(43) Pub. Date: Oct. 15, 2026

(54) NEUROSURGICAL DEVICE

(71) Applicant: NEUROCHASE TECHNOLOGIES LIMITED, Bristol (GB)

(72) Inventor: Steven Streatfield GILL, Bristol (GB)

(73) Assignee: NEUROCHASE TECHNOLOGIES LIMITED, Bristol (GB)

(21) Appl. No.: 19/484,678

(22) PCT Filed: May 10, 2024
(86) PCT No.: PCT/GB2024/051228

§ 371 Date: Nov. 14, 2025

(30) Foreign Application Priority Data
May 15, 2023 (GB) 2307199.6

(51) Int. Cl. A61M 5/14; A61M 25/00; A61M 39/08

(52) U.S. Cl. CPC A61M 5/14; A61M 39/08; A61M 2025/0059; A61M 2210/0693

(57) ABSTRACT

A fluid transfer tube (6) for insertion into living tissue to deliver an infusate. The fluid transfer tube comprises a proximal end, a distal end (18) for insertion into the tissue, a through-bore (52) configured to allow flow of the infusate from the proximal end to the distal end, and a plurality of reflux regions (54) in an outer surface of the fluid transfer tube. The reflux regions extend from the distal end of the fluid transfer tube along a length of the fluid transfer tube. The reflux regions are configured to allow flow of the infusate from the distal end of the fluid transfer tube towards the proximal end along the reflux regions when the distal end of the fluid transfer tube is inserted into the tissue. The fluid transfer tube is configured for atraumatic insertion into the tissue.

The present invention relates to apparatuses and methods for use in neurosurgery. Particularly, the invention relates to an apparatus used to deliver, and a method of delivering, therapeutic agents by infusion directly into living tissue such as the brain parenchyma.

Treatment of neurological diseases can be hindered by the presence of the blood-brain barrier. The blood-brain barrier can make it difficult to develop therapeutic agents that can be delivered from the systemic circulation into parts of the central nervous system such as the brain parenchyma. It can be desirable to deliver therapeutic agents to specific regions of the brain (‘brain volumes’ or ‘target volumes’). Obtaining an appropriate concentration of the therapeutic agent in a target volume whilst minimising exposure of the rest of the brain to the therapeutic agent is desirable, to reduce undesired side effects. It is also desirable to ensure that the therapeutic agent is distributed evenly through the target volume while minimising trauma to the tissue in the target volume.

Convection Enhanced Delivery (CED) is a method of targeted delivery of therapeutic agents to particular brain volumes by the controlled infusion of the therapeutic agent delivered into the brain parenchyma in a fluid using extremely small cannulas or tubing (often referred to in the art as micro-catheters). The cannulas have a port or ports at their distal end, allowing an infusate including the therapeutic agent to exit the catheter into the target brain volume. A continuous pressure gradient must be achieved at the port to overcome the local pressure of the target brain volume, thereby allowing the infusate to flow effectively into the target brain volume.

There are challenges in using CED because fluid flowing from a cannula port will tend to be trapped in a region directly around the cannula port by the surrounding tissue and the sealing of the tissue to the end of the cannula. To overcome this sealing and drive fluid into the tissue, the pressure of infusate can be increased. However, the increased pressure often fractures the tissue around the cannula port, causing trauma to the tissue and uneven or inadequate distribution of infusate following the fractures.

In addition, infusate is often delivered only into a small, approximately spherical or pear-shaped region extending from the cannula port or into a cavity in the tissue. To deliver therapy to clinically meaningful tissue volumes can therefore be problematic. Most targets for treating CNS (central nervous system) diseases by CED require several cannulas to be implanted to achieve the desired coverage of infusate into brain tissue. When the desired target volume and shape has been defined from MRI images, the number and orientation of cannulas required to fill the volume are determined by understanding the likely distribution shape and volume that can be achieved with the cannulas being deployed. Sequential infusions made at different points may be required along a trajectory into the brain to fill an elongate volume of tissue. Similarly, multiple passes may be required to fill a larger, more spherical structure in the brain.

In view of the aforementioned difficulties, there is still a need for a neurosurgical device that can provide improved control over the delivery of infusate to a target volume with reduced likelihood of causing trauma to the surrounding tissue.

According to a first aspect, there is provided a fluid transfer tube for insertion into living tissue to deliver an infusate, the fluid transfer tube comprising: a proximal end; a distal end for insertion into the tissue; a through-bore configured to allow flow of the infusate from the proximal end to the distal end; and a plurality of reflux regions in an outer surface of the fluid transfer tube, the reflux regions extending from the distal end of the fluid transfer tube along a length of the fluid transfer tube, wherein: the reflux regions are configured to allow flow of the infusate from the distal end of the fluid transfer tube towards the proximal end along the reflux regions when the distal end of the fluid transfer tube is inserted into the tissue; and the fluid transfer tube is configured for atraumatic insertion into the tissue.

By providing reflux regions extending along the fluid transfer tube, the distribution of the infusate into the surrounding tissue can be more accurately controlled. Infusate can also be effectively distributed over a much larger volume simultaneously, allowing for more rapid infusions at lower fluid pressure. This can reduce the length of procedures while also reducing the risk of damage to sensitive tissues such as the brain.

Optionally, the reflux regions are provided by variation in a radius of the fluid transfer tube, optionally wherein the radius of the fluid transfer tube is reduced in the reflux regions relative to the radius of the fluid transfer tube circumferentially adjacent to the reflux regions. Variation in the radius creates differences in the resistance to flow of infusate along the outer surface that can be used to create the reflux regions. Reduced radius in the reflux regions creates a clear path for the infusate to follow.

Optionally, an outer diameter of the fluid transfer tube and the variation in the radius of the fluid transfer tube permit the atraumatic insertion into the tissue. By choosing appropriate magnitude and profile of variations in the radius, the likelihood of damage to surrounding tissue during insertion can be reduced.

Optionally, the radius of the fluid transfer tube increases monotonically away from a centre of the reflux regions in a circumferential direction, optionally wherein the radius is strictly increasing away from the centre. A continuously increasing radius creates a single minimum position within each reflux region that improves consistency of reflux behaviour.

Optionally, the distal end of the fluid transfer tube is configured to compress the tissue when inserted into the tissue; and the compression of the tissue by the reflux regions is lower than the compression of the tissue by the outer surface of the fluid transfer tube circumferentially adjacent to the reflux regions. Lower compression of surrounding tissue by the reflux regions means that a less effective seal is formed around the fluid transfer tube and infusate can flow along that region of the tissue at lower fluid pressures. The reduced compression of tissue can also promote dispersion of infusate into the tissue above the reflux regions because intracellular spaces are more open in the tissue. This leads to improved dispersion of infusate into tissue, more homogeneous distribution, and more accurate control of infusate distribution at lower fluid pressures.

Optionally, the reflux regions are configured to allow the flow of the infusate along the reflux regions in preference to the infusate penetrating the tissue when the distal end of the fluid transfer tube is inserted into the tissue. This ensures that the infusate rapidly flows along the reflux regions so that infusate comes into contact with the tissue over the entire area into which infusate is to be delivered. This helps to ensure the infusate is delivered to the target volume rather than being pushed out into other nearby tissue.

Optionally, an outer diameter of the fluid transfer tube is no greater than 1 mm, optionally no greater than 0.75 mm, optionally no greater than 0.5 mm. Narrow tubes of these diameters are less intrusive and less likely to cause damage to surrounding tissue when inserted.

Optionally, the through-bore of the fluid transfer tube has a diameter of no greater than 0.4 mm, optionally no greater than 0.3 mm, optionally no greater than 0.2 mm. A narrow through-bore reduces constraints on the overall size of the fluid transfer tube and allows increased design flexibility for the outer surface of the fluid transfer tube.

Optionally, a minimum radius of the fluid transfer tube in the reflux regions is at least 50%, optionally at least 60%, optionally at least 70%, optionally at least 80% of a radius of the fluid transfer tube circumferentially adjacent to the reflux regions. This means that the variations in the radius of the fluid transfer tube are not too large, thereby reducing the stresses on surrounding tissue and the likelihood of damage to that tissue.

Optionally, a minimum radius of the fluid transfer tube in the reflux regions is at most 90%, optionally at most 80%, optionally at most 70%, optionally at most 60% of a radius of the fluid transfer tube circumferentially adjacent to the reflux regions. These differences in the radius in the reflux regions are large enough to clearly define the reflux regions and help to ensure that the desired reflux behaviour is created.

Optionally, the reflux regions are substantially longitudinal, said reflux regions optionally being parallel to the longitudinal axis of the fluid transfer tube. Longitudinal regions allow for rapid reflux of the infusate in the proximal direction to fill the intended volume.

Optionally, the reflux regions extend along at least a portion of the fluid transfer tube that is configured to be in contact with the tissue when the distal end of the fluid transfer tube is inserted into the tissue. This helps to ensure that infusate is effectively dispersed over the entire target volume of tissue.

Optionally, the reflux regions do not extend to the proximal end of the fluid transfer tube. This reduces the likelihood of leakage of the infusate out of the body during delivery of infusate.

Optionally, the plurality of reflux regions comprises at least 4 reflux regions, optionally at least 5 reflux regions, optionally at least 6 reflux regions, optionally 5 reflux regions in total. A larger number of regions helps to more evenly disperse the infusate around the fluid transfer tube and the target volume. Six reflux regions has been found to have a particularly good distribution of infusate around the fluid transfer tube without overly complicating the manufacturing process.

Optionally, the reflux regions comprise channels. Channels are straightforward to manufacture and provide a very clearly defined path of infusate to reflux

Optionally, an outer diameter of the fluid transfer tube and a depth of the channels permit the atraumatic insertion into the tissue. Appropriate depth of the channels can permit atraumatic insertion.

Optionally, the channels have a radial depth of at least 0.05 mm, optionally at least 0.075 mm, optionally at least 0.1 mm. These depths provide sufficient reduction in radius to promote infusate flow while being sufficiently shallow to permit atraumatic insertion.

Optionally, the outer surface of the fluid transfer tube in the reflux regions is concave, and/or the outer surface of the fluid transfer tube circumferentially adjacent to the reflux regions is convex. A concave reflux region helps to define a clear minimum radius point in the reflux region to improve consistency of refluxing behaviour. A convex profile adjacent to the reflux regions provides a smooth transition from the reflux region.

Optionally, the outer surface of the fluid transfer tube at the distal end is smoothly curved or flat at all points around the circumference of the fluid transfer tube. A smooth surface reduces the risk of the surface catching or cutting surrounding tissue and causing damage during insertion or use.

Optionally, a maximum curvature of the outer surface of the fluid transfer tube circumferentially adjacent to the reflux regions is no greater than a maximum curvature of the outer surface of the fluid transfer tube in the reflux regions. Ensuring that the maximum curvature is not at the regions of greater radius adjacent to the reflux regions reduces the strain on surrounding tissue when conforming to the outer surface.

Optionally, a perimeter of the fluid transfer tube is at least 5% larger, optionally at least 10% larger, optionally at least 15% larger than an outer circumference of the fluid transfer tube. This increases the surface area over which the infusate is in contact with the surrounding tissue.

Optionally, the outer surface of the fluid transfer tube is substantially flat in the reflux regions. Flat regions can be more straightforward to manufacture and provide a smooth profile that is unlikely to cause tissue damage.

Optionally, the plurality of reflux regions comprises a total of 6 reflux regions and the distal end of the fluid transfer tube has a hexagonal profile. A hexagonal profile has been found to provide a good balance of reflux performance and manufacturing simplicity.

Optionally, the fluid transfer tube is configured for insertion into the tissue through a guide tube comprising a proximal end and a distal end configured for insertion into tissue. A guide tube allows for accurate placement of the fluid transfer tube along a desired trajectory into the tissue and helps to prevent infusate from leaving the target volume.

According to a second aspect, there is provided a neurosurgical apparatus comprising: a guide tube comprising: a proximal end; and a distal end configured for insertion into tissue; and the fluid transfer tube of the first aspect configured for insertion into the tissue through the guide tube.

A guide tube allows for accurate placement of the fluid transfer tube along a desired trajectory into the tissue and helps to prevent infusate from leaving the target volume.

Optionally, the distal end of the fluid transfer tube is configured to project beyond a distal end of the guide tube into the tissue by a length that can be adjusted and/or fixed. This allows the size of the target volume to be adjusted for different procedures.

Optionally, the reflux regions do not extend along a portion of the fluid transfer tube that is configured to be within the guide tube when the distal end of the fluid transfer tube is inserted into the tissue. This reduces flow of infusate back up inside the guide tube, which may be undesirable and increase likelihood of leaks.

Optionally, the fluid transfer tube comprises a stop that engages with a proximal end of the guide tube and thereby sets the length by which the fluid transfer tube projects beyond the distal end of the guide tube. This allows the length of the target volume to be easily set before insertion of the guide tube and fluid transfer tube, thereby reducing the complexity of the insertion procedure. This decreases the likelihood of errors by users of the system.

Optionally, a difference between an outer diameter of the fluid transfer tube and an outer diameter of the distal end of the guide tube is such as to form a step that limits reflux of the infusate proximal to the step when the fluid transfer tube is inserted into the tissue through the through-bore of the guide tube. The step forms an effective seal that confines the infusate to the target volume.

According to a third aspect, there is provided a kit for convection-enhanced delivery of an infusate to tissue, said kit comprising: a fluid transfer tube comprising a proximal end, a distal end for insertion into the tissue, a through-bore configured to allow flow of the infusate from the proximal end to the distal end, and a plurality of reflux regions in an outer surface of the fluid transfer tube, the reflux regions extending from the distal end of the fluid transfer tube along a length of the fluid transfer tube; and a delivery probe configured for insertion into the tissue to form a fluid transfer tube track, wherein a cross-sectional area of the delivery probe is greater than a cross-sectional area of the distal end of the fluid transfer tube.

A kit with an appropriately designed delivery probe can ensure that dimensions of the probe and track formed are appropriately matched for the fluid transfer tube.

Optionally, the kit further comprises a guide tube comprising: a proximal end; and a distal end configured for insertion into tissue; and the fluid transfer tube is configured for insertion into the tissue through the guide tube. A guide tube allows for accurate placement of the fluid transfer tube along a desired trajectory into the tissue and helps to prevent infusate from leaving the target volume.

According to a fourth aspect, there is provided a method of convection-enhanced delivery of an infusate to tissue comprising: delivering the infusate into the tissue via a fluid transfer tube, wherein delivering the infusate comprises flowing the infusate from a distal end of the fluid transfer tube towards a proximal end of the fluid transfer tube along a plurality of reflux regions in an outer surface of the fluid transfer tube.

By providing reflux regions extending along the fluid transfer tube, the distribution of the infusate into the surrounding tissue can be more accurately controlled. Infusate can also be effectively distributed over a much larger volume simultaneously, allowing for more rapid infusions at lower fluid pressure. This can reduce the length of procedures while also reducing the risk of damage to sensitive tissues such as the brain.

Optionally, the method further comprises, prior to delivering the infusate: advancing a delivery probe into the tissue to form a fluid transfer tube track; inserting the fluid transfer tube into the tissue along the fluid transfer tube track. Using an appropriately designed delivery probe can ensure that dimensions of the probe and track formed are appropriately matched for the fluid transfer tube.

Optionally, the inserting of the fluid transfer tube is atraumatic. This avoids damage to surrounding tissue that could cause complications following the infusion procedure.

Optionally, a cross-sectional area of the distal end of the delivery probe is greater than a cross-sectional area of the distal end of the fluid transfer tube. This ensures that space is provided around the fluid transfer tube to allow for reflux of infusate.

Optionally, the method further comprises inserting a guide tube into the tissue, wherein: the advancing of the delivery probe comprises advancing the delivery probe through the guide tube; the fluid transfer tube track extends from the distal end of the guide tube; and the inserting of the fluid transfer tube comprises inserting the fluid transfer tube through the guide tube. Using the guide tube helps to guide the probe and fluid transfer tube along the desired trajectory.

Optionally, the method comprises initially delivering the infusate at a first flow rate, and subsequently delivering the infusate at a second flow rate, wherein the first flow rate is lower than the second flow rate. The initially lower flow rate helps to gradually adapt surrounding tissue to the inflow of fluid, thereby reducing risk of tissue fracture.

Optionally, the first flow rate is at most 1 μl/min. This has been found to be an appropriately low initial flow rate to avoid tissue fracture.

Optionally, the second flow rate is between 3 μl/min and 7 μl/min, optionally wherein the second flow rate is 3 μl/min or 5 μl/min. These flow rates allow for delivery of a clinically-effective volume of infusate in a reasonable timescale.

Optionally, the method comprises continuously increasing a rate of delivery of the infusate from the first flow rate to the second flow rate, optionally wherein the rate of delivery is increased by at most 0.5 μL/min², further optionally by at most 0.2 μL/min². Continuous increase avoids step changes that could cause high strain to surrounding tissue.

In any of the aspects, the tissue is optionally animal tissue, optionally mammalian tissue, optionally human tissue. Optionally, the tissue is brain tissue, optionally brain parenchyma. Optionally, the infusate is a therapeutic fluid. These tissues are particularly suited for treatment using these devices and methods because it may be more difficult to infuse therapeutic fluids using other devices or methods.

Embodiments of the present invention will now be described by way of non-limitative example with reference to the accompanying drawings, in which:

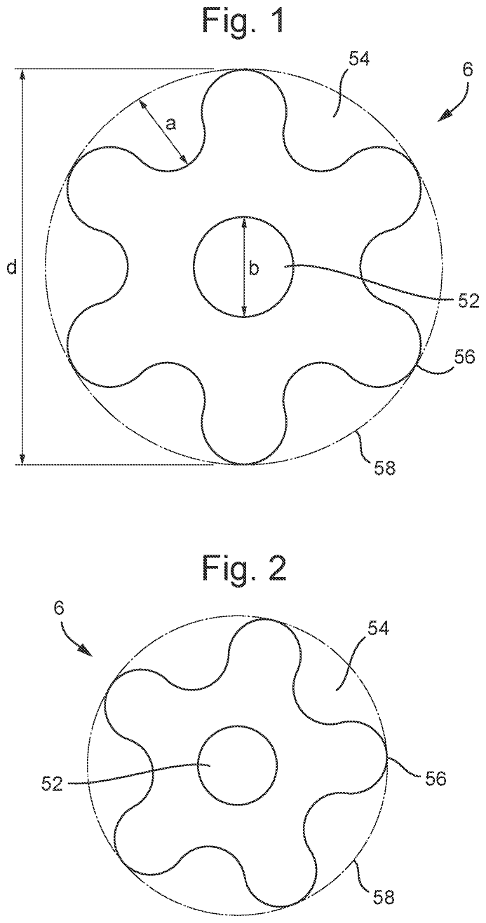

FIG. 1 is a cross-section of a fluid transfer tube with reflux regions;

FIG. 2 is a cross-section of a fluid transfer tube having five reflux regions

FIG. 3 is a view of the fluid transfer tube of FIG. 1 extending from a guide tube;

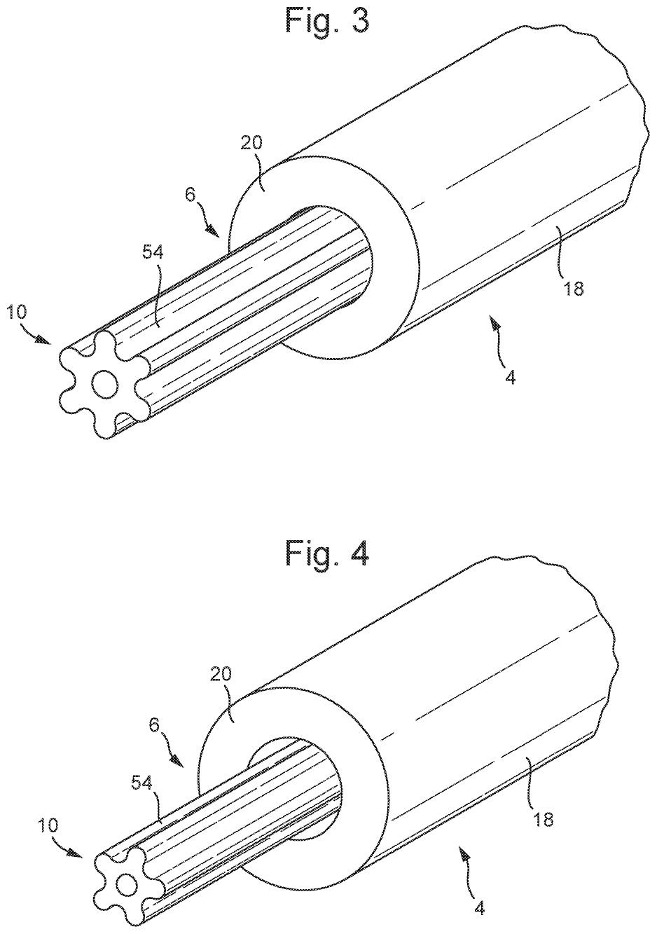

FIG. 4 is a view of the fluid transfer tube of FIG. 2 extending from a guide tube;

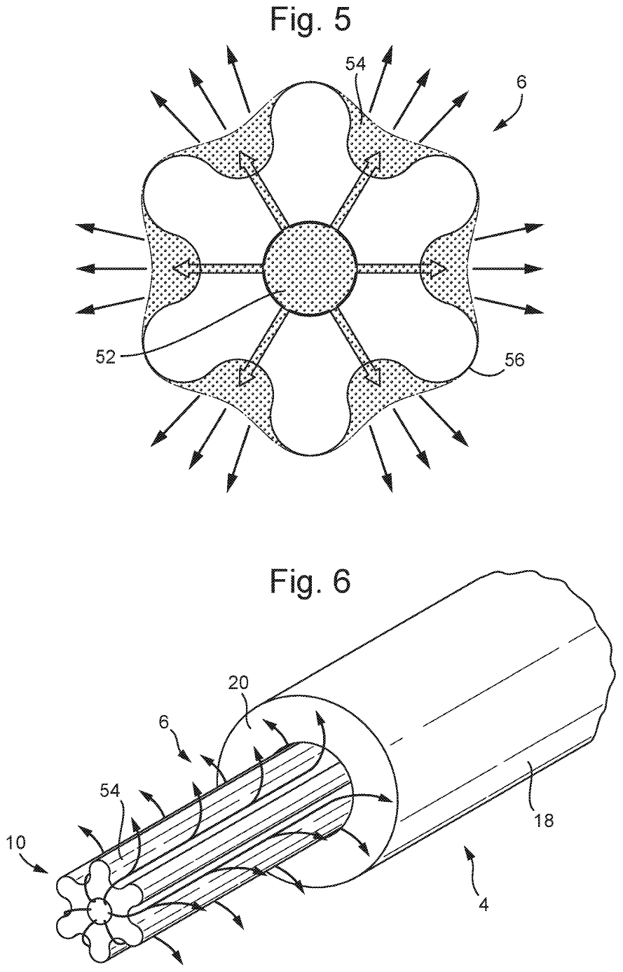

FIG. 5 is a cross-section of the tube of FIG. 1 demonstrating reflux of infusate;

FIG. 6 is a further view demonstrating reflux of infusate from the tube of FIG. 1;

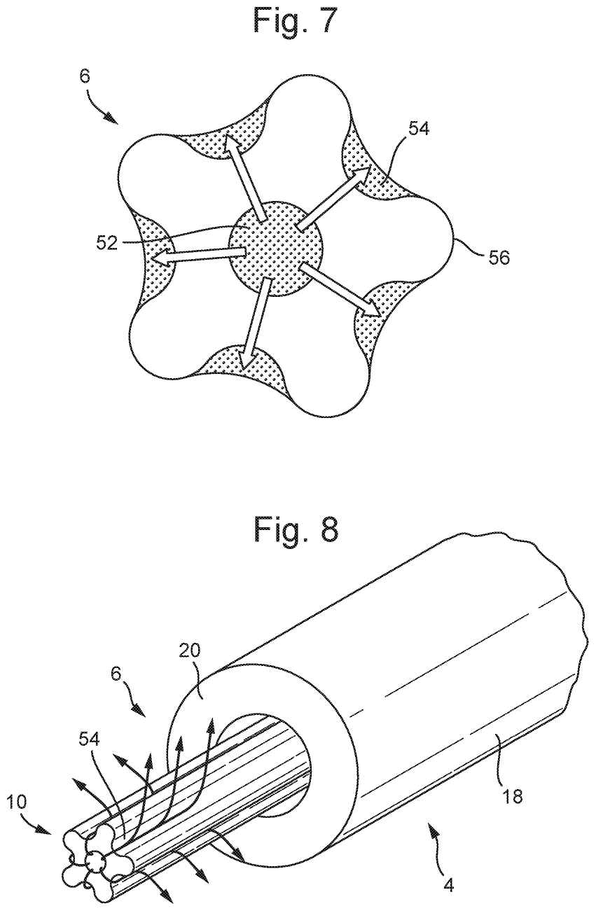

FIG. 7 is a cross-section of the tube of FIG. 2 demonstrating reflux of infusate;

FIG. 8 is a further view demonstrating reflux of infusate from the tube of FIG. 2;

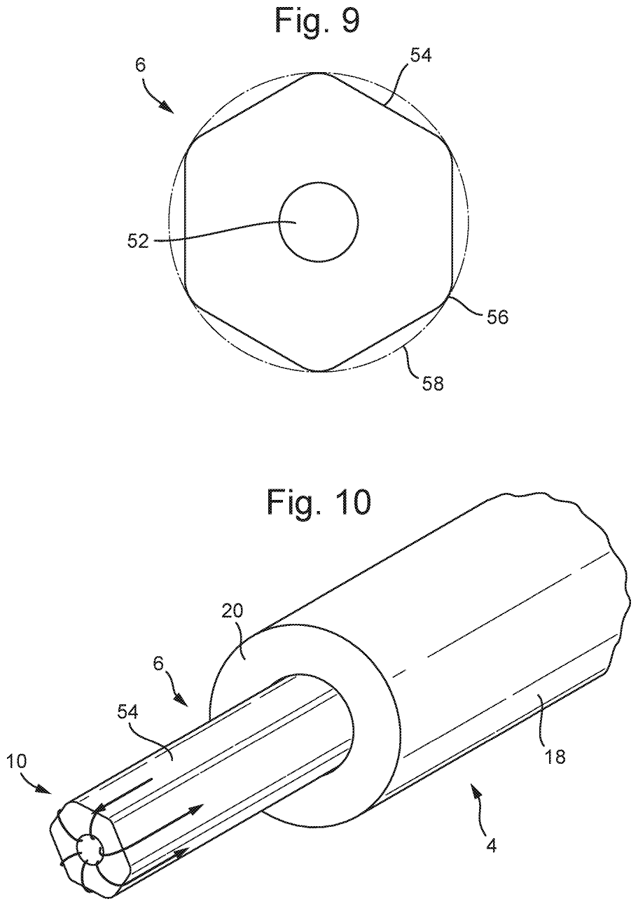

FIG. 9 is a cross-section of an alternative design for a fluid transfer tube with reflux regions; and

FIG. 10 is a view of the fluid transfer tube of FIG. 9 extending from a guide tube.

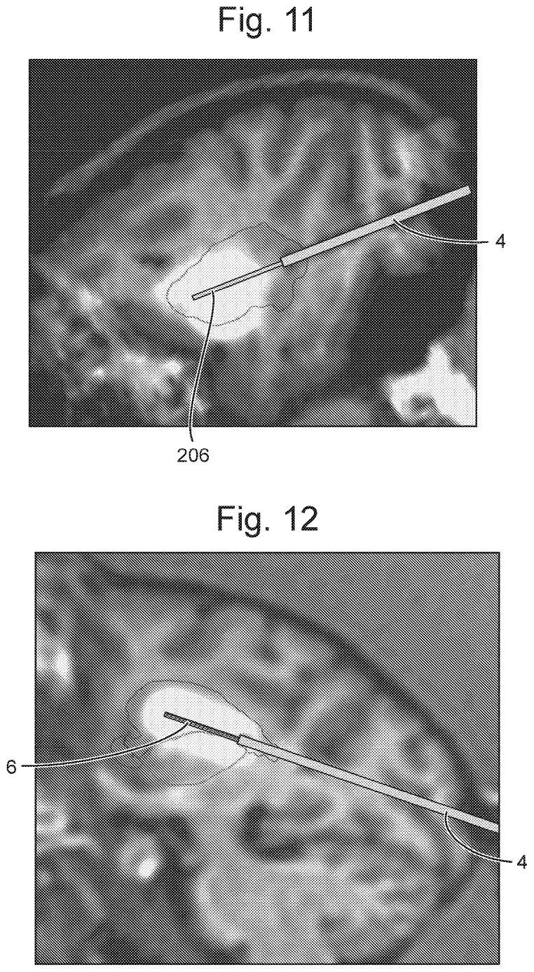

FIG. 11 is an MRI image taken during delivery of infusate using a prior art fluid transfer tube;

FIG. 12 is an MRI image taken during delivery of infusate using the fluid transfer tube of FIG. 1;

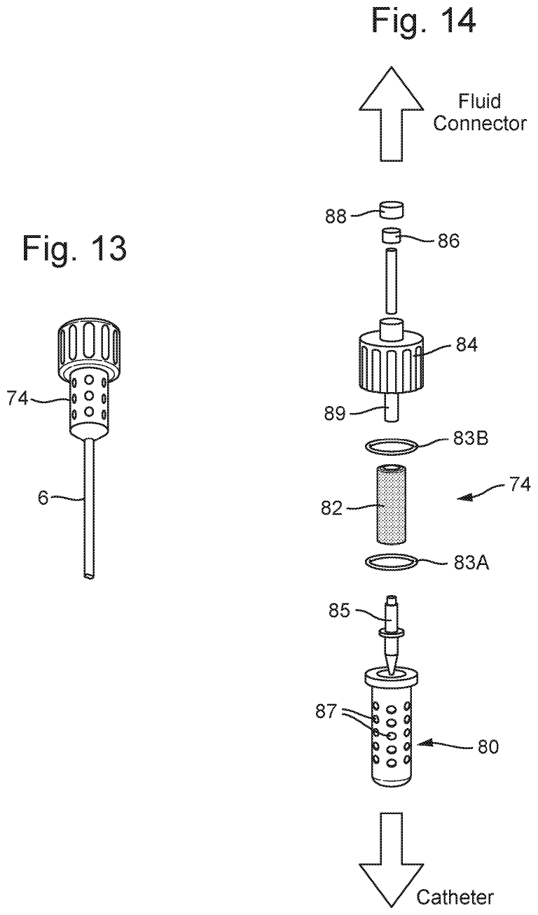

FIG. 13 shows a bubble vent;

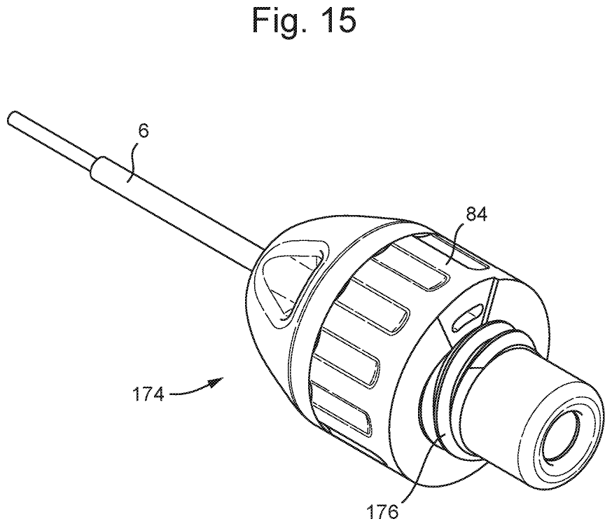

FIG. 14 is an exploded view of the bubble vent of FIG. 13;

FIG. 15 shows another design of bubble vent;

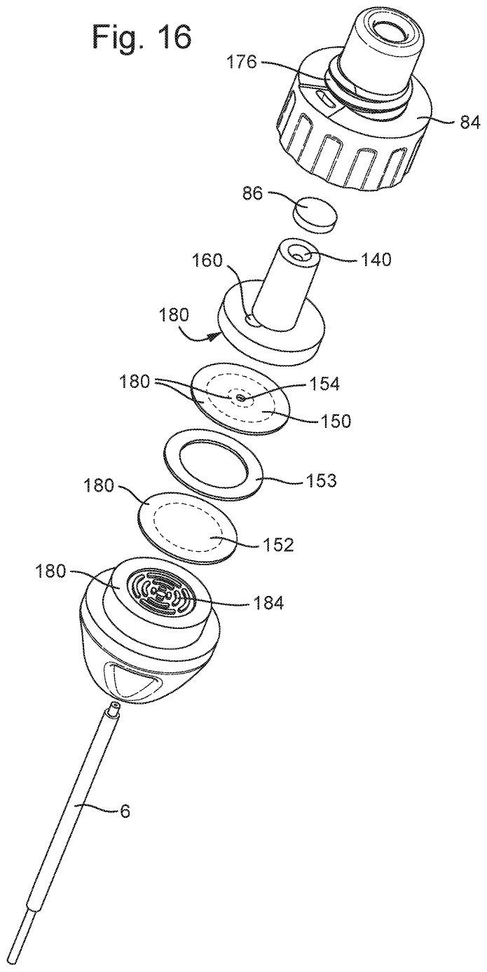

FIG. 16 is an exploded view of the bubble vent of FIG. 15;

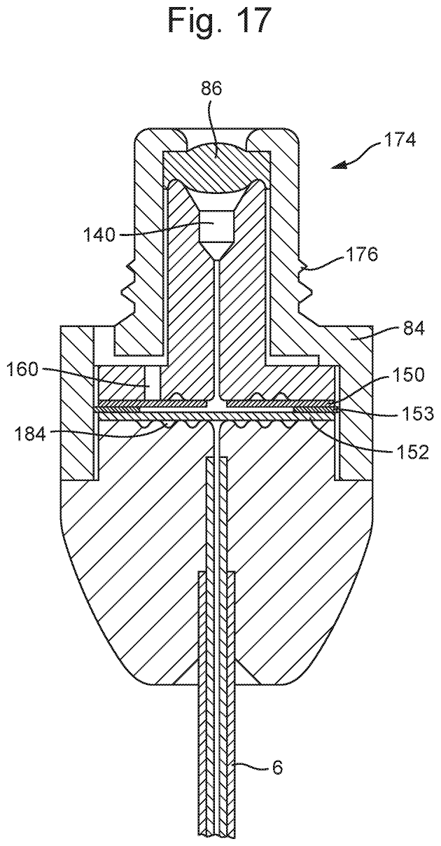

FIG. 17 is a cross-sectional view of the bubble vent of FIG. 15;

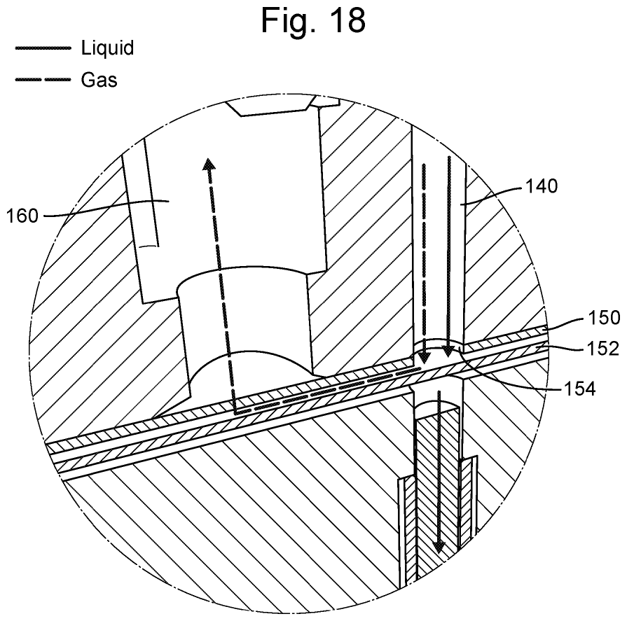

FIG. 18 shows the operation of the bubble vent of FIG. 15;

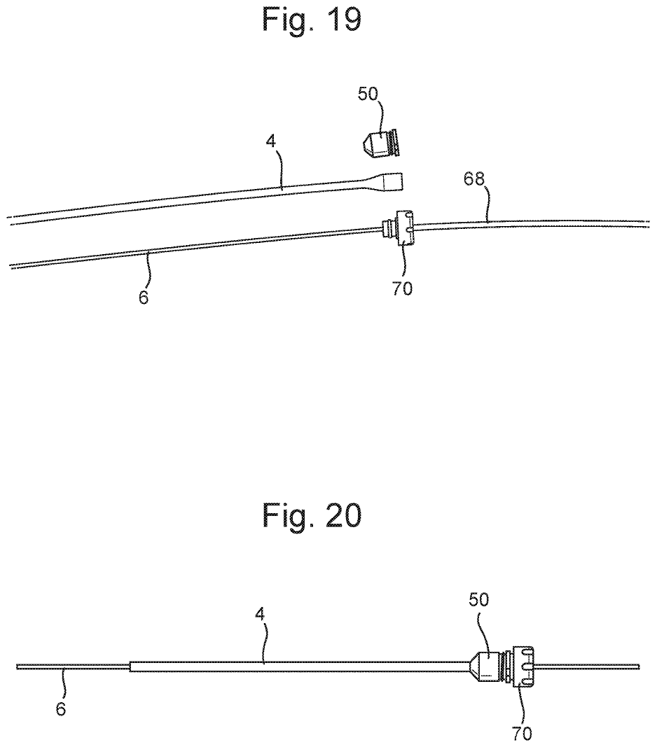

FIG. 19 shows a neurosurgical apparatus comprising a fluid transfer tube;

FIG. 20 shows the neurosurgical apparatus of FIG. 19 assembled;

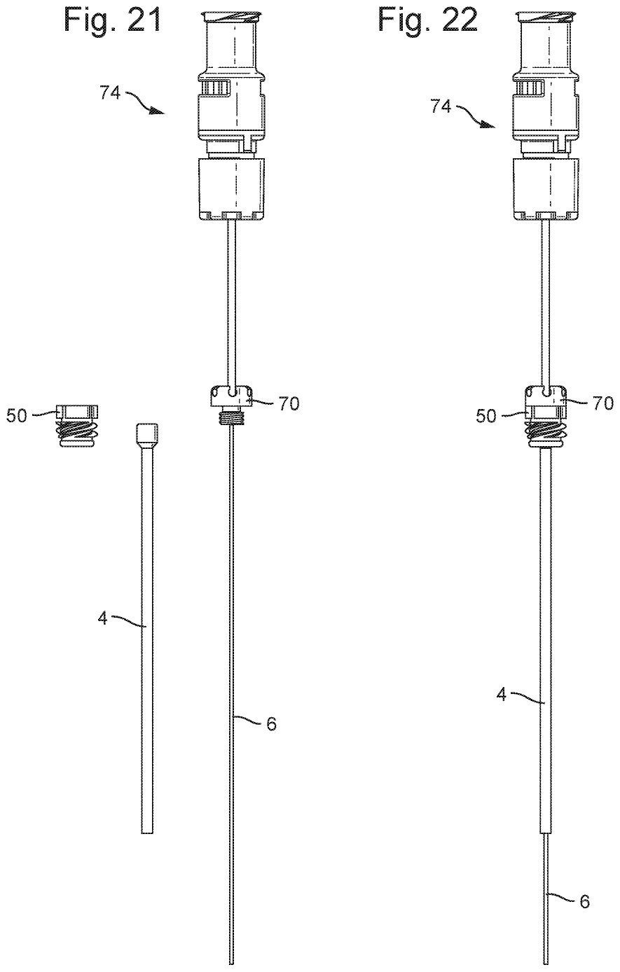

FIG. 21 shows a neurosurgical apparatus comprising a fluid transfer tube and integral bubble vent; and

FIG. 22 shows the neurosurgical apparatus of FIG. 21 assembled.

To address the limitations of prior art devices as described above, the present disclosure provides a fluid transfer tube 6 such as that shown in cross-section in FIG. 1. The fluid transfer tube 6 is for insertion into living tissue to deliver an infusate. The tissue may be animal tissue, for example a mammalian tissue such as human tissue. In particular, the tissue may be brain tissue such as brain parenchyma. The fluid transfer tube 6 (which may also be referred to as a cannula or catheter) may therefore be used for providing fluid access to, for example to permit fluid transfer to or from, the central nervous system of a mammal.

Primarily, the fluid transfer tube 6 is intended for providing fluid access to the brain of a human. In other words, the fluid transfer tube 6 is configured to permit fluid transfer to or from the brain of a mammal, and may be configured to deliver the infusate using convection enhanced delivery. In particular, the fluid transfer tube 6 is used to transfer fluid to or from a target brain volume. As discussed above, treatment of neurological diseases using therapeutic agents can be hindered by the blood-brain barrier, so treatment using a fluid transfer tube 6 is advantageous for these diseases. The infusate may be a therapeutic fluid. The infusate may carry any suitable therapeutic agent, inert fluid, imaging agent or diagnostic agent that can be delivered into the tissue via a suitable biologically inert fluid.

The fluid transfer tube 6 may be configured for insertion into the tissue through a guide tube 4 comprising a proximal end and a distal end 18 configured for insertion into tissue. The fluid transfer tube 6 is passed along a through-bore of the guide tube from the proximal end of the guide tube to the distal end 18 of the guide tube 4. The fluid transfer tube 6 typically projects beyond the distal end 18 of the guide tube 4 so that the distal end 10 of the fluid transfer tube 6 is positioned at the brain target volume. The guide tube 4 and fluid transfer tube 6, optionally together with other components described below, may be referred to as a cannula assembly, neurosurgical cannula assembly, or neurosurgical apparatus.

The fluid transfer tube 6 is configured to be inserted into the tissue coaxial with a trajectory to a target, for example within the brain. The trajectory may be established with image guidance, and may be defined between an entry point, for example on the skull, and the target. The target may be a particular region of or location in the brain of the mammal. The distal end 10 of the fluid transfer tube 6 may be inserted to a predetermined location in the brain along the trajectory to the target.

The fluid transfer tube 6 comprises a proximal end, and a distal end 10 for insertion into the tissue. The proximal end of the fluid transfer tube 6 may be located, in use, outside the brain (although may still be within the skull and/or cranial cavity). The fluid transfer tube 6 may have a fluid connector at its proximal end configured for connecting to a fluid infusion line or directly to a syringe and infusion pump. At least the distal end 10 of the fluid transfer tube 6 may be configured for insertion into the tissue using image guidance. For example, at least the distal end 10 of the fluid transfer tube 6 may comprise a radiopaque material. Optionally, the fluid transfer tube 6 may comprise a radiopaque material along substantially the entire length of the fluid transfer tube 6.

The fluid transfer tube 6 comprises a through-bore 52 configured to allow flow of the infusate from the proximal end to the distal end 10. The through-bore of the fluid transfer tube 6 may have a diameter b of no greater than 0.4 mm, optionally no greater than 0.3 mm, optionally no greater than 0.2 mm. The through-bore of the fluid transfer tube 6 may have a diameter b of between 0.1 mm and 0.4 mm, for example 0.4 mm. Optionally, the through-bore of the fluid transfer tube 6 may have a diameter b between 0.10 mm and 0.15 mm, for example approximately 0.127 mm (five thousandths of an inch).

The length of the fluid transfer tube 6 may be at least 100 mm. In general, the fluid transfer tube 6 may have a length at least that of the guide tube 4 that the fluid transfer tube 6 is configured for use with.

The fluid transfer tube 6 may comprise a biocompatible material. For example, the fluid transfer tube 6 may comprise a biocompatible plastic such as polyetheretherketone (PEEK) or a polyurethane such as carbothane. The fluid transfer tube 6 may be formed from a rigid material, for example fused silica, ceramic, for example zirconia ceramic, a metal such as titanium or stainless steel, or a stiff biocompatible plastic such as PEEK. Alternatively, the fluid transfer tube 6 may be formed from a flexible material, for example a flexible biocompatible plastic. The choice of whether to use a rigid or flexible fluid transfer tube 6 may be made based on the specific application. Optionally, the distal end 10 of the fluid transfer tube 6 is rounded, conical, or bullet shaped to minimise tissue trauma upon its insertion. However, this is not essential and the distal end 10 may be square-cut. Due to the atraumatic insertion properties of the fluid transfer tube 6, for example due to its narrow outer diameter, the fluid transfer tube 6 does not cause significant tissue damage on insertion. The shape of the distal end 10 is therefore less important to reduce trauma.

To improve lubricity, reduce tissue adherence, reduce tissue integration capability,, or provide other desirable properties, at least an outer surface 56 of the fluid transfer tube 6 may comprise a coating. For example, the outer surface 56 may be post processed with, for example, plasma treatment or have an appropriate coating applied to provide it with the desired properties. For example, a lubricious coating of Parylene or polytetrafluoroethylene (PTFE) may be used to reduce shear forces on tissue when the fluid transfer tube 6 is inserted into the tissue. Alternatively, a lubricious heat shrink plastic tube may be applied to the outer surface of the fluid transfer tube 6. For example, the heat shrink tube may be a formed of PTFE, FEP, PET, or Polyolefin.

As another example, a coating may be provided at the distal end 10 of the fluid transfer tube to reduce tissue adherence or integration, for example a cytotoxic coating. For chronic implantation of fluid transfer tubes 6, there is a chance that tissue may grow or attach around the distal end 10, thereby blocking the throughbore 52 and preventing delivery of infusate. An appropriate coating can prevent or reduce this effect.

A material and/or thickness of the fluid transfer tube 6 may be such that the tube can be cut by hand using a knife or sharp blade. The fluid transfer tube 6 may be cut before insertion to the desired insertion length with respect to a fixation of its proximal end, for example at the skull. The fluid transfer tube 6 may be cut with a sharp blade in a cutting jig.

The fluid transfer tube 6 comprises a plurality of reflux regions 54 in an outer surface 56 of the fluid transfer tube 6. The plurality of reflux regions 54 may comprise at least 4 reflux regions 54, optionally at least 5 reflux regions 54, optionally at least 6 reflux regions 54. As shown in FIG. 1, the plurality of reflux regions may comprise 6 reflux regions 54 in total, i.e. exactly 6 reflux regions 54.

In another embodiment shown in FIG. 2, the plurality of reflux regions may comprise 5 reflux regions 54 in total, i.e. exactly 5 reflux regions 54, such that the plurality of reflux regions consists of exactly 5 reflux regions 54. Using 5 reflux regions may be preferable because 5 reflux regions has been found to provide superior distribution than 4 or 6 reflux regions. This is due to the balance between the number of reflux regions 54 around the fluid transfer tube 6 and the size of each individual reflux region 54, which can be larger with 5 reflux regions 54 than with 6 reflux regions.

The reflux regions 54 extend from the distal end 10 of the fluid transfer tube 6 along a length of the fluid transfer tube 6. The reflux regions 54 may be substantially longitudinal. For example, the reflux regions 54 may be parallel to the longitudinal axis of the fluid transfer tube 6, as shown for the 6 reflux region embodiment in FIG. 3 and the 5 reflux region embodiment in FIG. 4. However, this is not essential, and other configurations are possible. For example, the reflux regions 54 may extend helically along the length of the fluid transfer tube 6 around the outer surface 56 from the distal end 10. Each reflux region 54 forms a continuous region in the outer surface 56.

The reflux regions 54 are configured to allow flow of the infusate from the distal end 10 of the fluid transfer tube 6 towards the proximal end of the fluid transfer tube 6 along the reflux regions 54 when the distal end 10 of the fluid transfer tube 6 is inserted into the tissue. This provides a more uniform and consistent distribution of the infusate around the fluid transfer tube 6. The reflux regions 54 also reduce the area of contact with the tissue, thereby increasing the lubricity of the fluid transfer tube 6 and ease of insertion into the tissue.

The effect of the reflux regions 54 is demonstrated for the 6 reflux region embodiment in FIG. 5 and FIG. 6, and for the 5 reflux region embodiment in FIG. 7 and FIG. 8. Infusate flows down the through-bore 52 from the proximal end of the fluid transfer tube and out of the through-bore 52 at the distal end 10 of the fluid transfer tube 6. The reflux regions 54 provide regions of lower resistance to flow of fluid, so that infusate flows back around the fluid transfer tube 6 along the interface of the outer surface 56 with the tissue towards the proximal end. Infusate can then be forced outwards into the surrounding tissue along the entire length of the reflux regions 54 that are in contact with the tissue.

The reflux regions 54 may be configured to allow the flow of the infusate along the reflux regions 54 in preference to the infusate penetrating the tissue when the distal end 10 of the fluid transfer tube 6 is inserted into the tissue. The infusate will therefore initially flow along the outer surface 56 of the fluid transfer tube 6 as shown in FIG. 6 and FIG. 8 to fill the reflux regions 54 before moving outward into the surrounding tissue. This means that infusate can be distributed more evenly over the entire area of the outer surface 56 that is in contact with the tissue.

As mentioned above, the fluid transfer tube 6 may be used in combination with a guide tube 4. The guide tube 4 has a larger outer diameter than the fluid transfer tube 6. A difference between an outer diameter d of the fluid transfer tube 6 and an outer diameter of the distal end 18 of the guide tube 4 is such as to form a step 20. The step 20 limits reflux of the infusate proximal to the step 20 when the fluid transfer tube 6 is inserted into the tissue through the through-bore of the guide tube 4.

When infusate is driven down the fluid transfer tube 6 and into the tissue through its distal end 10, the refluxing infusate will travel back along the reflux regions 54 towards the step 20 created by the change in diameter at the distal end 18 of the guide tube 4 from which the fluid transfer tube 6 extends. A high-pressure zone in the tissue just distal to the step 20 created by insertion of the guide tube 4 will compress the tissue interface and resist reflux outside of the target region.

A further advantage of the reflux regions 54 is that infusate can be distributed over a larger volume at lower pressure. The low resistance of the reflux regions 54 causes the infusate to be distributed more easily over the outer surface 56 of the fluid transfer tube 6. In prior art devices, compression of the surrounding tissue can form a tight seal around the outer surface of the fluid transfer tube. This is especially true for devices intended for atraumatic insertion, for example that have narrow outer diameters. This means that increasing liquid pressure is required to drive the infusate out from the distal end into the tissues. Once the infusate is expelled from the distal end 10, it will be unable to reflux back up the outer surface of the device due to the effective sealing of the tissue around the outer diameter. This means that the infusate will tend to be forced outwards in a spherical region centred on the distal end of the device. This provides limited control of the volume over which infusate is delivered. The higher fluid pressures also create a relatively high risk of tissue damage or of infusate being forced out of the target region altogether into other areas of tissue.

In contrast in the present fluid transfer tube 6, the reflux regions 54 provide a low-resistance path for infusate to move away from the distal end 10 of the fluid transfer tube 6 along the outer surface of the fluid transfer tube 6. This means that a lower liquid pressure can be used to distribute the infusate. It also means that the distance between the step 20 and the distal end 10 of the fluid transfer tube 6 can be used to more precisely and effectively control the volume over which infusate is delivered.

The fluid transfer tube 6 is configured for atraumatic insertion into the tissue. This means that the configuration of the fluid transfer tube 6 is such that insertion of the fluid transfer tube 6 into the tissue minimises injury, trauma, or damage to the tissue. For example, the configuration may be such that no tissue fracturing or tearing occurs on insertion. The insertion of the fluid transfer tube 6 may only dilate the surrounding tissue. Typically, as is discussed further below, a fluid transfer tube track is formed in the tissue prior to insertion of the fluid transfer tube 6. Atraumatic insertion may mean that no further injury, trauma, or damage to the tissue (such as tissue fracturing or tearing) occurs when the fluid transfer tube 6 is inserted into the tissue along the pre-formed fluid transfer tube track.

The configuration of the fluid transfer tube 6 for atraumatic insertion is provided by the suitable dimensions and properties of the fluid transfer tube 6 and reflux regions 54.

An outer diameter d of the fluid transfer tube 6 and the variation in the radius of the fluid transfer tube 6 permit the atraumatic insertion into the tissue while maintaining reflux regions. The outer diameter d of the fluid transfer tube 6 may be chosen to be sufficiently small to minimise tissue damage when inserted. The outer diameter may be no greater than 1.0 mm, optionally no greater than 0.75 mm, optionally no greater than 0.5 mm. The fluid transfer tube 6 may have an outer diameter of between 0.4 mm and 0.7 mm, preferably between 0.5 mm and 0.6 mm, for example 0.5 mm. For clarity, unless specified otherwise, the “outer diameter” of the fluid transfer tube 6 is generally used to mean an outermost or largest diameter of the fluid transfer tube 6, as denoted by the length d in FIG. 1.

The distal end of the fluid transfer tube 6 may be configured to laterally compress the tissue when inserted into the tissue, and the compression of the tissue by the reflux regions 54 may be lower than the compression of the tissue by the outer surface 56 of the fluid transfer tube 6 circumferentially adjacent to the reflux regions 54. This reduced compression of tissue by the reflux regions 54 leads to a lower liquid pressure being required for infusate to be forced along the reflux regions 54.

The reflux regions 54 may be provided by variation in a radius of the fluid transfer tube 6. In the below, the radius of the fluid transfer tube 6 will generally be defined for a particular point on the outer surface 56 of the fluid transfer tube 6 and unless specified otherwise refers to a distance from the central longitudinal axis of the fluid transfer tube 6 to that point.

For example, as in FIG. 1, the radius of the fluid transfer tube 6 may be reduced in the reflux regions 54 relative to the radius of the fluid transfer tube 6 circumferentially adjacent to the reflux regions 54. The reflux regions 54 may comprise regions in which the radius of the fluid transfer tube 6 is reduced relative to a maximum radius of the fluid transfer tube 6. In FIG. 1, each reflux region 54 immediately abuts the circumferentially adjacent reflux regions 54 on either side, such that the maximum radius of the fluid transfer tube 6 is only reached at the points on the outer surface where two adjacent reflux regions 54 meet. However, this is not in general essential, and in other implementations the reflux regions 54 may be spaced apart circumferentially around the outer surface 56 of the fluid transfer tube 6. In particular, the reflux regions 54 may be evenly spaced around a circumference of the fluid transfer tube 6.

A cross-sectional profile of each of the plurality of reflux regions 54 may be the same. This helps to provide consistent and predictable reflux behaviour around the entire fluid transfer tube 6.

The radius of the fluid transfer tube 6 may increase monotonically away from a centre of the reflux regions 54 in a circumferential direction. That is, each reflux region 54 may have a unique point of minimum radius. The radius may be strictly increasing away from the centre of the reflux region 54.

A minimum radius of the fluid transfer tube 6 in the reflux regions may be at least 50%, optionally at least 60%, optionally at least 70%, optionally at least 80% of a radius of the fluid transfer tube 6 circumferentially adjacent to the reflux regions 54 or of a maximum radius of the fluid transfer tube 6. A minimum radius of the fluid transfer tube 6 in the reflux regions is at most 90%, optionally at most 80%, optionally at most 70%, optionally at most 60% of a radius of the fluid transfer tube 6 circumferentially adjacent to the reflux regions 54 or of a maximum radius of the fluid transfer tube 6.

Each reflux region 54 has a width in the circumferential direction as well as extending along the length of the fluid transfer tube 6. The width in the circumferential direction of each reflux region 54 may be defined as an angular width in terms of the angle subtended by the reflux region 54 at the longitudinal axis of the fluid transfer tube 6. The angular width of each reflux region may be at least 15 degrees, optionally at least 30 degrees, optionally at least 45 degrees. The angular width of each reflux region may be at most 90 degrees, optionally at most 75 degrees, optionally at most 60 degrees. For example, the angular width of each reflux region may be 60 degrees as in FIG. 1. The width in the circumferential direction of each reflux region 54 may also be expressed as a distance around the circumference. The width may be at least 0.1 mm, optionally at least 0.15 mm, optionally at least 0.2 mm, optionally at least 0.25 mm.

The reflux regions 54 may extend along at least a portion of the fluid transfer tube 6 that is configured to be in contact with the tissue when the distal end 10 of the fluid transfer tube 6 is inserted into the tissue. This may be a portion of the fluid transfer tube 6 that projects beyond the distal end 18 of the guide tube 4 when the fluid transfer tube 6 is configured for insertion through a guide tube 4. The reflux regions 54 may not extend to the proximal end of the fluid transfer tube 6. The reflux regions 54 may extend at least 1 mm, optionally at least 2 mm, optionally at least 5 mm, optionally at least 10 mm from the distal end 10 of the fluid transfer tube 6.

However, in some implementations, the reflux regions 54 may extend to the proximal end of the fluid transfer tube 6 and/or may extend along substantially the entire length of the fluid transfer tube 6. This may be preferred depending on the method of manufacture of the fluid transfer tube 6. For example, manufacturing the fluid transfer tube 6 to have reflux regions 54 along its entire length, e.g. to have a uniform cross-section along its length, may reduce cost.

As shown in FIG. 1, the reflux regions 54 may comprise channels. In this case, the outer diameter d of the fluid transfer tube 6 and a depth of the channels may permit the atraumatic insertion into the tissue. The channels may have a radial depth a of at least 0.05 mm, optionally at least 0.075 mm, optionally at least 0.1 mm.

The outer surface 56 of the fluid transfer tube 6 in the reflux regions 54 may be concave as shown in FIG. 1. This defines a minimum radius in the reflux region 54 that will help to direct the flow of infusate. The outer surface 56 of the fluid transfer tube 6 circumferentially adjacent to the reflux regions 54 may be convex. The outer surface 56 circumferentially adjacent to the reflux regions 54 may in particular have a radius of curvature within 10%, optionally within 5%, optionally approximately equal to half the outer diameter d of the fluid transfer tube 6. A maximum curvature of the outer surface 56 of the fluid transfer tube 6 circumferentially adjacent to the reflux regions 54 may be no greater than a maximum curvature of the outer surface 56 of the fluid transfer tube 6 in the reflux regions 54. This prevents the fluid transfer tube 6 from having sharp tips or vanes around its circumference that may cause tissue damage on insertion.

The outer surface 56 of the fluid transfer tube 6 at the distal end 10 may be smoothly curved or flat at all points around the circumference of the fluid transfer tube 6. This also helps to prevent tissue damage on insertion, particular because twisting of the fluid transfer tube 6 may be possible when the tube is inserted or secured at its proximal end.

A perimeter of the fluid transfer tube 6 may be at least 5% larger, optionally at least 10% larger, optionally at least 15% larger than an outer circumference of the fluid transfer tube 6. This helps to increase the surface area over which infusate is distributed during use. Outer circumference here refers to a circumference defined by the outer diameter d of the fluid transfer tube 6, as denoted by the dashed circle 58 in FIG. 1.

FIG. 9 and FIG. 10 show an alternative design for a fluid transfer tube 6. In this design, the outer surface 56 of the fluid transfer tube 6 is substantially flat in the reflux regions 54. The cross-section of the fluid transfer tube 6 may therefore be that of a regular polygon. The vertices of the regular polygon in this case may be rounded to reduce the likelihood of trauma to surrounding tissue. In FIG. 9, the plurality of reflux regions 54 comprises a total of 6 reflux regions 54, such that the distal end 10 of the fluid transfer tube 6 has a hexagonal profile in cross-section.

FIG. 11 and FIG. 12 are MRI images taken during infusion procedures to demonstrate the effectiveness of the present fluid transfer tube 6. In both cases, a fluid transfer tube 6, 206 is inserted into brain tissue along a guide tube 4.

FIG. 11 shows the left putamen of a non-human primate following infusion of 150 μl of infusate containing an MRI contrast agent (gadoteridol) using a prior art fluid delivery tube 206 that lacks reflux regions 54. The infusate was delivered at 1 μl/min for 10 mins, 2 μl/min for 5 mins, and then at 3 μl/min for 43 mins 20 seconds. The bright white area shows where infusate has entered the tissue surrounding the distal end 10 of the fluid transfer tube 6. As can be seen, the infusate has escaped out of the target volume of the 3 mm cylinder shown in red. The infusate has instead formed an irregular spherical distribution roughly centred on the distal end of the tube 206. The infusate has travelled much further in the direction towards the bottom of the image than in the opposite direction towards the top of the image. This is likely due to the infusate leaking out of the target brain structure into surrounding white matter, which has a lower resistance to fluid flow. This loss of containment makes it impossible to accurately deliver the infusate, because any further increase of fluid pressure will cause the infusate to continue leaking into surrounding tissue, rather than refluxing up the tube to fill the target volume.

FIG. 12 shows infusion into a putamen using a fluid transfer tube 6 of the present disclosure according to the design shown in FIG. 1. In this case, it can be seen that the bright white area of infusate extends from the distal end 10 of the fluid transfer tube 6 proximally to the step where the distal end of the guide tube is located. The infusate assumes a well-defined regular cylindrical shape around the distal end 10 of the fluid transfer tube 6 where the fluid transfer tube 6 projects beyond the end of the guide tube 4. This demonstrates how the reflux regions 54 allow for more consistent and controlled infusate delivery.

It is important that air not be delivered into the brain through the fluid transfer tube 6, and to reduce this risk, the fluid transfer tube 6 may comprise a bubble vent 74. The bubble vent 74 is preferably provided at a proximal end of the fluid transfer tube 6, as shown in FIG. 13. The configuration and component parts of the bubble vent 74 are described below and illustrated in FIG. 14. The bubble vent 74 is configured to prevent gas from entering the fluid transfer tube 6. The bubble vent 74 mitigates the risk of bubbles entering the brain if they have come out of solution in the infusate or have been entrained in the infusate during the connection and/or disconnection of a delivery system that is used to deliver the infusate to the fluid transfer tube 6, such as a dispenser, infusion line, and/or pump. Bubbles infused into brain tissue can tear the tissue and disrupt distribution of the therapeutic fluid/infusate. The bubble vent 74 may be permanently joined to and/or integrally formed with the fluid transfer tube 6, which further reduces the chance of bubbles being entrained during connection of the fluid transfer tube to the delivery system. The bubble vent 74 is preferably provided integrally with the cannula 6, as shown in Fig. 13. FIG. 14 illustrates an exploded view of the bubble vent 74, configured for attachment to the cannula 6 and a fluid connector of the delivery system. The bubble vent 74 includes a perforated filter guard 80, a bubble filter 82, retaining rings 83A, 83B, a retaining cap 84, a septum stopper 86 and a septum cap 88. The bubble vent 74 may comprise a low volume bubble filter 82, for example made from expanded polytetrafluoroethylene (ePTFE). The bubble filter 82 may have a hydrophobic (optionally superhydrophobic), gas permeable, microporous structure configured to remove bubbles from the flowing therapeutic fluid/infusate. The bubble filter 82 may be effective to remove bubbles at flow rates of less than or equal to 30 μl/min. The bubble vent 74 may further comprise a filter guard 80. In the illustrated example the bubble filter 82 is effective to remove bubbles at flow rates of less than or equal to 30 μl/min. The filter 82 is contained in the perforated filter guard 80 and in the illustrated example the bubble filter 82 is received over a hollow post 85 and is retained by a retainer ring 83A to the hollow post 85, which is positioned concentric to the filter guard 80.

The filter guard 80 may comprise a hollow shell that includes a distribution of a plurality of perforations 87 (small holes) around the shell wall that facilitate degassing the fluid/infusate as it flows through the bubble vent 74 to the fluid transfer tube 6. The filter guard 80 may also guard/protect the bubble filter 82 against damage. The combination of the bubble filter 82 and the filter guard 80 facilitates dispersion of entrained air/bubbles from the fluid flow before the fluid passes into the fluid transfer tube 6.

The bubble 74 vent may comprise a retaining cap 84, which connects with the filter guard 80 to complete the assembly of the bubble vent 74 and to contain the bubble filter 82 within the bubble vent 74. The retaining cap 84 may include a hollow retaining post 89 and a retainer ring 83B which engage with the bubble filter 82 to ensure the bubble filter 82 is correctly positioned and retained in the filter guard 80 to ensure efficient functionality of the bubble filter 82 during use. The retaining cap 84 may include a septum stopper 86, which provides a sealed unit until the septum 86 is pierced by a hollow needle to provide fluid connection to the fluid transfer tube 6. The septum stopper 86 may be retained under compression by a septum cap 88. The retaining cap 84 and filter guard 80 may be joined by a snap fit connection. However, alternative arrangements could be used to join them together, for example a threaded connection, welded connection, glued connection etc.

The bubble vent 74 incorporating the bubble filter 82 reduces the risk of air being delivered e.g. to the brain with the fluid containing therapeutic agent/infusate. It will be appreciated fluid containing air/bubbles will be space occupying and therefore is capable of stretching and tearing brain tissue whilst also disrupting delivery/distribution of the therapeutic agent/infusate.

FIGS. 15 to 18 show an alternative design of bubble vent 174. The bubble vent 174 is configured to prevent gas from entering the fluid transfer tube 6. The bubble vent 174 may additionally or alternatively be configured to prevent pathogens (for example micro-organisms such as bacteria) from entering the fluid transfer tube 6. This reduces the risk of intracranial infections occurring due to the treatment.

FIG. 15 shows the bubble vent 174 in its assembled state ready for use. Similar to the bubble vent 74, the bubble vent 174 comprises a retaining cap 84. The retaining cap 84 comprises a proximal connector 176, for example a threaded connector, for connection to a fluid connector of the delivery system.

FIG. 16 shows an exploded view of the bubble vent 174, and FIG. 17 shows a cross-sectional view. The proximal connector 176 may comprise a septum 86 to seal the proximal connector 176 until the septum 86 is pierced, for example by a hollow needle. The bubble vent 174 comprises a fluid passage 140 fluidly connecting the proximal connector 176 and the fluid transfer tube 6. In this design, the bubble vent comprises a first membrane 150 and a second membrane 152. The first membrane 150 and the second membrane 152 are positioned between a distal end of the fluid passage 140 and a proximal end of the fluid transfer tube 6. The first membrane 150 and the second membrane 152 may be substantially parallel to one another, and may be substantially perpendicular to an axis of the fluid passage 140. The first membrane 150 is positioned closer to the distal end of the fluid passage 140 than the second membrane 152, such that fluid entering the bubble vent 174 through the septum 86 reaches the first membrane 150 before the second membrane 152. An annular washer 153 may be positioned between the first membrane 150 and the second membrane 152 that forms a peripheral fluid seal between the membranes and the housing of the connector 174 and separates the membranes centrally to create a cylindrical gap between them. The cylindrical gap may have a diameter of between 2 mm and 6 mm but is most preferably 4 mm. The gap may separate the membranes 150 and 152 by 0.05 mm to 0.2 mm but most preferably by 0.1 mm. The first membrane 150 and the second membrane 152 may be connected to one another and to the other components of the bubble vent 174 via connection surfaces 180. The connection surfaces 180 may be joined by any suitable method, for example using ultrasonic welding or using an adhesive layer. The bubble vent 174 may comprise a support member 184 to support the distal surface of the second membrane 152 and allow liquid that has passed through the second membrane 152 to more easily reach the cannula 6.

The first membrane 150 is hydrophobic and gas permeable. A hole 154 is provided in the first membrane 150 where the fluid passage 140 meets the first membrane 150, such that fluid from the fluid passage 140 can pass through the first membrane 150 via the hole 154. The septum sealed connector 174 may comprise a support member to support the proximal surface of the first membrane 150 and annular connection surfaces to attach the membrane around its periphery and around its central hole 154 (not shown in FIG. 16). The second membrane 152 is liquid permeable and preferably hydrophilic. It is not essential that the second membrane 152 is hydrophilic, however gas venting works most efficiently using the combination of a hydrophobic and a hydrophilic membrane. Use of the hydrophobic first membrane 150 alone could result in air being drawn from the atmosphere through the first membrane 150 and into the infusate if the pressure in the line falls below atmospheric pressure. This can happen if the connector is elevated above the head by more than 10-25 cm (depending on intracranial pressure). The second membrane 152 being hydrophilic prevents air ingress into the brain even in such situations. The second membrane 152 is impermeable to gas and bacteria. No hole is provided in the second membrane 152, such that fluid from the fluid passage 140 must pass through the material of the second membrane 152 to reach the fluid transfer tube 6. One or more vent holes 160 (for example, two vent holes in the example of FIG. 16) are provided in the bubble vent 174 on a proximal side of the first membrane 150. No holes are provided in the first membrane 150 where the vent holes 160 meet the first membrane 150, such that fluid from the fluid passage 140 must pass through the material of the first membrane 150 to reach the vent holes 160.

The operation of the bubble vent 174 is demonstrated in the close-up view of FIG. 18. A mixture of liquid and gas (for example an infusate to be delivered to a patient's brain via the fluid transfer tube 6 with some entrained bubbles of air) enters the bubble vent 174 via the septum 86 and the fluid passage 140. The mixture passes through the first membrane 150 via the hole 154. The liquid is drawn to the hydrophilic second membrane 152, and soaks through the second membrane 152 (which is liquid permeable) into the cannula. The layer of liquid and the second membrane 152 form a barrier preventing gas from passing into the fluid transfer tube 6. The gas will pass along the air gap between the first membrane 150 and the second membrane 152, and can escape through the gas-permeable first membrane 150 at the position of one of the vents 160. The hydrophobic nature of the first membrane 150 repels liquid and prevents the liquid forming a similar barrier as on the second membrane 152, thereby allowing the gas to pass out of the bubble vent 174 via the vent holes 160.

The fluid transfer tube 6 may be provided as part of a neurosurgical apparatus that also comprises a guide tube 4, such as the neurosurgical apparatus of FIG. 19. The guide tube 4 comprises a proximal end and a distal end 18. The distal end 18 is configured for insertion into tissue. The guide tube 4 may be configured for insertion into the tissue using image guidance. For example, at least a distal end 18 of the guide tube 4 may comprise a radiopaque material. The guide tube 4 comprises a through-bore 30 for passage of the fluid transfer tube 6. The guide tube 4 (or the cannula assembly where the fluid transfer tube 6 is inserted into the guide tube 4 prior to insertion into the tissue) is preferably passed into the tissue down a pre-made track. As the guide tube 4 enters the track, the track is gently dilated up to the larger diameter of the guide tube 4. The fluid transfer tube 6 is then inserted into the tissue through a through-bore of the guide tube 4.

The distal end 10 of the fluid transfer tube 6 may be configured to project beyond a distal end 18 of the guide tube 4 into the tissue by a length that can be adjusted and/or fixed. The fluid transfer tube 6, when inserted through the through-bore of the guide tube 4 prior to its insertion in the tissue, may be positioned so that its distal end 10 is at least aligned with the distal end 18 of the guide tube 4 or projects beyond it. The guide tube 4 may be inserted into the brain tissue with the fluid transfer tube 6 projecting beyond its distal end 18 by a predetermined length so that when the distal end 18 of the guide tube 4 reaches the target region, the distal end 10 of the fluid transfer tube 6 will also reach its planned target. Alternatively, after insertion of the guide tube 4 the distal end 10 of the fluid transfer tube 6 may be advanced through the tissue to the selected target.

When used for fluid access to a brain, the guide tube 4 may be inserted through a burr hole in the skull that has already been fitted with a guide hub 50. The guide hub 50 is for securing to the skull of a patient before insertion of the guide tube 4 into the brain. The separate guide hub may be provided as part of the neurosurgical apparatus comprising the guide tube 4 and the fluid transfer tube 6 and/or other components. The guide hub 50 has a passage for the guide tube 4 therethrough. The guide hub 50 provides a fixed datum point at the skull, from which the probes, guide tube 4, and fluid transfer tube 6 direction and length can be directed. The proximal end of the guide tube 4 can be secured to the guide hub 50, for example by a fitting such as a screw fitting with a bore therethrough. Interposition of a deformable washer, such as a silicone washer, between the distal end of the screw fitting and the guide tube 4 may radially compress and seal around the fluid transfer tube 6. This may also fix the position of the fluid transfer tube 6 with respect to the guide hub 50 and thereby the skull. In compressing around the fluid transfer tube 6, such a washer can also create a fluid seal between the fluid transfer tube 6 and the guide tube 4.

The fluid transfer tube 6, in particular a proximal end of the fluid transfer tube 6, may comprise a stop 70 that engages with a proximal end of the guide tube 4 and thereby sets the length by which the fluid transfer tube 6 projects beyond the distal end of the guide tube 4. The stop 70 may also be configured to engage with a proximal end of the guide tube 4 to form a fluid seal between the fluid transfer tube 6 and the guide tube 4. This seals a potential leak path of liquid that could otherwise reflux between the through-bore of the guide tube 4 and the fluid transfer tube 6 to the outside of the tissue. The seal also closes a path of potential bacterial ingress into the tissue. The fluid seal may be formed by compression of a deformable washer, such as a silicone washer, or by compression of a resiliently deformable outer layer of the guide tube 4.

The position of the stop 70 along the length of the fluid transfer tube 6 may be adjustable. This allows for the length of the fluid transfer tube 6 that protrudes beyond the distal end 18 of the guide tube 4 to be adjusted. Alternatively, the stop 70 may be fixed to the fluid transfer tube 6 and the depth of insertion of the fluid transfer tube 6 adjusted by cutting it to the desired length with respect to the stop 70 using a knife. The stop 70 may be a threaded stop which is screwed into a complimentary thread within the bore of the guide hub.

The stop 70 may be overmoulded onto the fluid transfer tube 6. The stop 70 may comprise indentations or castellations on its proximal surface configured to receive and retain the fluid transfer tube 6. The fluid transfer tube 6 may be bent and press-fitted into the indents to hold its proximal portion at an angle to the portion that is inserted into the tissue. This can help to direct the fluid transfer tube 6 outside of the tissue.

The reflux regions 54 may extend along a portion of the fluid transfer tube 6 proximal of the stop 70, for example if the reflux regions 54 extend to the proximal end of the fluid transfer tube 6 and/or extend along substantially the entire length of the fluid transfer tube 6. In this case, a sleeve 68 may be provided over a portion of the fluid transfer tube 6 proximal of the stop 70, i.e. over a portion of the fluid transfer tube configured to be outside of the tissue when the distal end 10 of the fluid transfer tube 6 is inserted into the tissue.

The function of the sleeve 68 may be to protect the fluid transfer tube 6 or help to retain the fluid transfer tube 6 in the indents or castellations of the stop 70. The sleeve 68 may be fixed to the fluid transfer tube 6 as part of the attachment of the stop 70 to the fluid transfer tube 6. For example, the sleeve 68 may be placed on the fluid transfer tube 6 before the stop 70 is attached to the fluid transfer tube 6. A distal end of the sleeve 68 may be secured inside the stop 70, for example during overmoulding of the stop. The sleeve 68 may comprise a plastic material. The sleeve 68 may be attached to the fluid transfer tube 6 by heat shrinking.

The reflux regions 54 may not extend along a portion of the fluid transfer tube 6 that is configured to be within the guide tube 4 when the distal end 10 of the fluid transfer tube 6 is inserted into the tissue. Therefore, only the length of the fluid transfer tube 6 that is configured to project beyond the distal end 18 of the guide tube 4 into the tissue may comprise the reflux regions 54. This reduces the likelihood of infusate travelling back up the inside of the guide tube 4 towards the proximal end of the guide tube 4, which may cause undesirable loss of liquid or increase the likelihood of leaks outside the target region.

The guide tube 4 may be a rigid guide tube that is inserted to a predetermined location in the tissue along a trajectory that is established with image guidance. A rigid guide tube will generally be used with a rigid fluid transfer tube 6. The rigid guide tube comprises a rigid material, for example ceramic, a metal such as titanium or stainless-steel, or a rigid plastic material such as PEEK. The proximal end of a rigid guide tube is generally guided by and held during use by a stereoguide or robot guide.

Convection enhanced delivery techniques can be used for both acute (short time) and chronic (longer term or repeated) delivery of treatment to tissue. For example, gene therapy may be carried out in a single treatment session, whereas treatments for other reasons such as chemotherapy may require repeated infusions into the brain (a chronic treatment regime).

When the guide tube 4 is rigid, it can be inserted directly into the tissue without the need to form a track in the tissue beforehand. A rigid guide tube 4 and corresponding rigid fluid transfer tube 6 may be particularly suited for acute treatments, where the guide tube 4 is inserted into the brain to perform a particular procedure and then removed once the procedure is complete.

When a rigid guide tube 4 is used, the fluid transfer tube 6 may be inserted into the through-bore 30 of the guide tube 4 before the guide tube 4 is inserted into the tissue, so that the guide tube 4 and fluid transfer tube 6 are inserted into the tissue as a single unit (cannula assembly). In this case, in use the fluid transfer tube 6 is primed with a liquid prior to insertion of the cannula assembly into the tissue. This prevents air being trapped in the fluid transfer tube 6 that would then be forced out into the tissue when liquid is passed into the fluid transfer tube 6. The fluid transfer tube 6 may be positioned and retained within the through-bore of the guide tube by a releasable compression seal at the proximal end of the guide tube 4. The compression seal may interact with the depth-controlling stop 70 on the fluid transfer tube 6, if present. The fluid transfer tube 6 may also be rigid. For example, the fluid transfer tube 6 may comprise or be formed from a rigid material such as fused silica, a metal such as titanium or stainless steel, or a stiff biocompatible plastic such as polyetheretherketone (PEEK).

Alternatively, the guide tube 4 may be a cuttable or flexible guide tube, that is cut to a prescribed length and whose distal end is inserted to a predetermined location in the tissue with image guidance and whose proximal end is fixed in the skull during use. A material and/or thickness of the guide tube 4 may be such that the guide tube 4 can be cut by hand using a knife or sharp blade. When the guide tube 4 is flexible, the fluid transfer tube 6 may also be flexible. For example, the fluid transfer tube 6 may comprise or be made from a flexible material. This allows the fluid transfer tube 6 to move and retain its intended position at the target if movement of the tissue occurs.

An example neurosurgical apparatus is shown in FIG. 19 and FIG. 20. The neurosurgical apparatus of FIG. 19 and FIG. 20 comprises the fluid transfer tube 6, guide tube 4, guide hub 50, and stop 70. FIG. 19 shows the components of the apparatus separately, and FIG. 20 shows the components assembled together as they would be during use.

An further example neurosurgical apparatus is shown in FIG. 21 and FIG. 22. The neurosurgical apparatus of FIG. 21 and FIG. 22 is substantially similar to the apparatus of FIG. 19 and FIG. 20, but also comprises a bubble vent 74 provided integrally with the proximal portion of the fluid transfer tube 6. FIG. 21 shows the components of the apparatus separately, and FIG. 22 shows the components assembled together as they would be during use.

In contrast to holding the guide tube 4 by a stereoguide during the delivery of an infusate, where only a single target infusion can be accomplished at a time, use of the embodiment with bone fixation using a guide hub 50 facilitates infusion through several implanted devices simultaneously. This greatly reduces procedure time and the risk to patients. Fixing the proximal end of the guide tube 4 to the skull (or to a guide hub 50 that is fixed to the skull) provides a relatively secure arrangement for long duration or chronic treatments. This type of guide tube 4 can therefore be left in place for extended periods of time for use when chronic, or chronic intermittent infusions are required.

In some situations, for example when using a rigid guide tube 4, the fluid transfer tube 6 and guide tube 4 may be assembled together and delivered into the tissue together. In other situations, for example when using guide tubes 4 such as those in FIG. 19 and FIG. 20 that are secured to the skull, the distal end 10 of the fluid transfer tube 4 may be passed into the through-bore of the guide tube 4 after insertion of the guide tube 4 into the tissue, passing through the proximal end of the guide tube 4 to the distal end 18 and being delivered to the target. During the passage of the fluid transfer tube 6 through the through-bore of the guide tube 4, a column of air in the guide tube 4 is forced distally with a piston-like action. This can lead to tearing of tissue in the target region. To mitigate this, the guide tube 4 may be configured to vent gas from the through-bore of the guide tube 4 to a proximal end of the guide tube 4, preferably through an inner surface of the through-bore. Venting of air from the guide tube 4 to atmosphere may be stopped when the fluid transfer tube 6 is fully inserted into the guide tube 4. At this point, the fluid transfer tube 6, for example via the stop 70 or an interposing washer, may engage with the proximal end of the guide tube 4 to create a seal.

The fluid transfer tube 6 may also be provided as part of a kit for convection-enhanced delivery of an infusate to tissue. The kit comprises the fluid transfer tube 6 comprising a proximal end, a distal end 10 for insertion into the tissue, a through-bore 52 configured to allow flow of the infusate from the proximal end to the distal end 10, and the plurality of reflux regions 54 in the outer surface 56 of the fluid transfer tube 6. The kit may also comprise the guide tube 4 and/or the guide hub 50.

The kit further comprises a delivery probe configured for insertion into the tissue to form a fluid transfer tube track. The delivery probe may be a track forming probe.

The delivery probe may be configured for insertion into the through-bore of the guide tube 4. The guide tube 4 may be inserted into the tissue when mounted on the delivery probe, or may be inserted into the guide tube 4 after the guide tube 4 is already inserted. Once the distal end 18 of the guide tube 4 is located at its pre-determined target location in the tissue, the delivery probe may be advanced further along the same trajectory as the guide tube 4. The distal end of the delivery probe may be advanced until it reaches the planned location for the fluid transfer tube 6, forming a track for the fluid transfer tube 6. The delivery probe is then withdrawn, leaving the guide tube 4 in situ and a distal track to accommodate the fluid transfer tube 6. A distal end of the delivery probe may be configured for dissecting tissue to aid with forming the fluid transfer tube track.

A cross-sectional area of the delivery probe is greater than a cross-sectional area of the distal end 10 of the fluid transfer tube 6. This means that the tissue will not be greatly compressed where it contacts the fluid transfer tube 6 when the fluid transfer tube 6 is inserted. This reduces the sealing around the fluid transfer tube 6 and allows the infusate to more easily reflux along the reflux regions 54.

The delivery probe may comprise a rod having a distal end that is rounded or conical in shape. The rod may be provided with a spike extending axially from the distal end of the rod to provide the tissue-dissecting capability of the distal end of the delivery probe. The spike may have a narrower diameter than the rod. The spike may be provided with a rounded extreme distal end. The spike may taper from the point at which it joins the rod at the rounded distal end of the rod to the extreme distal end of the spike. The spike may have a maximum diameter that is at most, preferably less than, the diameter of the fluid transfer tube 6. In use, the spike at the distal end of the delivery probe can dissect tissue with reduced trauma as it is inserted, thereby creating a track with minimal tissue trauma.

The outer diameter of the distal end of the delivery probe may be approximately equal to the outer diameter of the fluid transfer tube 6. In this case, the reduced cross-sectional area of the delivery probe relative to the fluid transfer tube 6 is due to the reduction in cross-sectional area by the presence of the reflux regions 54. This choice of outer diameter also means that the surrounding tissue may be slightly compressed circumferentially adjacent to the reflux regions 54, so that a small sealing effect is achieved in these regions. This helps to ensure that the infusate initially flows along the reflux regions 54 in preference to diffusing into the surrounding tissue.

The delivery probe may be made from a rigid material such as hardened stainless steel or tungsten carbide. The delivery probe may have a coating to increase its lubriciousness, for example a coating of PTFE or Parylene. The profile of the delivery probe is designed to pass through tissue to a target with precision and minimal trauma so that the subsequent insertion of the fluid transfer tube is relatively atraumatic. It is also designed to form the track by gentle dilation of the tissue to allow for creation of a tissue seal, rather than fracturing of the tissue.

Each one of the guide tube 4, delivery probe, and the fluid transfer tube 6 may include any of the features described herein with respect to the other aspects as appropriate.

Methods of surgery making use of the fluid transfer tube 6 described herein are described below.

A method of convection-enhanced delivery of an infusate to tissue comprises delivering the infusate into the tissue via the fluid transfer tube 6. Delivering the infusate comprises flowing the infusate from a distal end 10 of the fluid transfer tube 6 towards a proximal end of the fluid transfer tube 6 along a plurality of reflux regions 54 in an outer surface 56 of the fluid transfer tube 6. The reflux regions 54 may be configured as described above in relation to the fluid transfer tube 6.

The method may further comprise, prior to delivering the infusate: advancing a delivery probe into the tissue to form a fluid transfer tube track; and inserting the fluid transfer tube 6 into the tissue along the fluid transfer tube track. A cross-sectional area of a distal end of the delivery probe is greater than a cross-sectional area of the distal end of the fluid transfer tube.

The inserting of the fluid transfer tube 6 may be atraumatic. This can be achieved with suitable choice of the configuration and dimensions of the fluid transfer tube 6 and the reflux regions 54 as described above.

The method may further comprise inserting a guide tube 4 into the tissue, wherein: the advancing of the delivery probe comprises advancing the delivery probe through the guide tube 4. The fluid transfer tube track may extend from the distal end of the guide tube 4, and the inserting of the fluid transfer tube 6 may comprise inserting the fluid transfer tube 6 through the guide tube 4.

The method may further comprise providing a positive pressure of the infusate within the fluid transfer tube 6 during the inserting of the fluid transfer tube 6. This helps to prevent coring of brain tissue by the fluid transfer tube 6, and also prevents entraining of air that could occur if the fluid transfer tube 6 was inserted while filled with gas. However, this is not essential, and alternatively the fluid transfer tube 6 may be primed or filled with infusate at neutral pressure prior to the inserting of the fluid transfer tube 6. While positive pressure can reduce coring in some situation, it also means that additional infusate is delivered during insertion of the fluid transfer tube 6 and prior to the distal end 10 of the fluid transfer tube 6 reaching the target region. This may be undesirable in some situations.

The method may comprise initially delivering the infusate at a first flow rate, and subsequently delivering the infusate at a second flow rate, wherein the first flow rate is lower than the second flow rate. The purpose of using the lower flow rate initially is to gradually open the interstitial space between cells in the tissue to allow fluid flow into the tissues without fracturing the tissue. This prevents a fracture forming, which will tend to propagate as the infusion proceeds if it does form. The first flow rate may be at most 1 μl/min. The second flow rate may be between 3 μl/min and 7 μl/min, optionally wherein the second flow rate is 3 μl/min or 5 μl/min.

The method may comprise continuously and/or smoothly increasing a rate of delivery of the infusate from the first flow rate to the second flow rate. The rate of delivery may be increased by at most 0.5 μL/min², further optionally by at most 0.2 μL/min².

Details of the method may also be described by the following numbered clauses.

M1. A method of convection-enhanced delivery of an infusate to tissue comprising: delivering the infusate into the tissue via a fluid transfer tube, wherein delivering the infusate comprises flowing the infusate from a distal end of the fluid transfer tube towards a proximal end of the fluid transfer tube along a plurality of reflux regions in an outer surface of the fluid transfer tube.

M2. The method of clause M1, wherein the method further comprises, prior to delivering the infusate: advancing a delivery probe into the tissue to form a fluid transfer tube track; inserting the fluid transfer tube into the tissue along the fluid transfer tube track.

M3. The method of clause M2, wherein the inserting of the fluid transfer tube is atraumatic.

M4. The method of clause M2 or M3, wherein a cross-sectional area of a distal end of the delivery probe is greater than a cross-sectional area of the distal end of the fluid transfer tube.

M5. The method of any of clauses M1 to M4, wherein the method further comprises inserting a guide tube into the tissue, wherein: the advancing of the delivery probe comprises advancing the delivery probe through the guide tube; the fluid transfer tube track extends from the distal end of the guide tube; and the inserting of the fluid transfer tube comprises inserting the fluid transfer tube through the guide tube.

M6. The method of any of clauses M1 to M5, wherein the method comprises initially delivering the infusate at a first flow rate, and subsequently delivering the infusate at a second flow rate, wherein the first flow rate is lower than the second flow rate.

M 7. The method of clause M 6, wherein the first flow rate is at most 1 μl/min.

M8. The method of clauses M6 or M7, wherein the second flow rate is between3 μl/min and 7 μl/min, optionally wherein the second flow rate is 3 μl/min or 5 μl/min.

M9. The method of any of clauses M6 to M8, wherein the method comprises continuously increasing a rate of delivery of the infusate from the first flow rate to the second flow rate, optionally wherein the rate of delivery is increased by at most 0.5 μL/min², further optionally by at most 0.2 μL/min².

M10. The method of any of clauses M1 to M9, wherein the tissue is animal tissue, optionally mammalian tissue, optionally human tissue.

M11. The method of any of clauses M1 to M10, wherein the tissue is brain tissue, optionally brain parenchyma.

M12. The method of any of clauses M1 to M11, wherein the infusate is a therapeutic fluid.

1-42. (canceled)

43. A fluid transfer tube for insertion into living tissue to deliver an infusate, the fluid transfer tube comprising:
a proximal end;
a distal end for insertion into the tissue;
a through-bore configured to allow flow of the infusate from the proximal end to the distal end; and
a plurality of reflux regions in an outer surface of the fluid transfer tube, the reflux regions extending from the distal end of the fluid transfer tube along a length of the fluid transfer tube, wherein:
the reflux regions are configured to allow flow of the infusate from the distal end of the fluid transfer tube towards the proximal end along the reflux regions when the distal end of the fluid transfer tube is inserted into the tissue; and
the fluid transfer tube is configured for atraumatic insertion into the tissue.

44. The fluid transfer tube according to claim 43, wherein the reflux regions are provided by variation in a radius of the fluid transfer tube, wherein the radius of the fluid transfer tube is reduced in the reflux regions relative to the radius of the fluid transfer tube circumferentially adjacent to the reflux regions.

45. The fluid transfer tube according to claim 44, wherein the radius of the fluid transfer tube increases monotonically away from a centre of the reflux regions in a circumferential direction, optionally wherein the radius is strictly increasing away from the centre.

46. The fluid transfer tube according to claim 43, wherein:
the distal end of the fluid transfer tube is configured to compress the tissue when inserted into the tissue;
the compression of the tissue by the reflux regions is lower than the compression of the tissue by the outer surface of the fluid transfer tube circumferentially adjacent to the reflux regions; and
the reflux regions are configured to allow the flow of the infusate along the reflux regions in preference to the infusate penetrating the tissue when the distal end of the fluid transfer tube is inserted into the tissue.

47. The fluid transfer tube according to claim 43, wherein one or more of:
a) an outer diameter of the fluid transfer tube is no greater than 1 mm, optionally no greater than 0.75 mm, optionally no greater than 0.5 mm;
b) the through-bore of the fluid transfer tube has a diameter of no greater than 0.4 mm, optionally no greater than 0.3 mm, optionally no greater than 0.2 mm;
c) a minimum radius of the fluid transfer tube in the reflux regions is at least 50%, optionally at least 60%, optionally at least 70%, optionally at least 80% of a radius of the fluid transfer tube circumferentially adjacent to the reflux regions; or
d) a minimum radius of the fluid transfer tube in the reflux regions is at most 90%, optionally at most 80%, optionally at most 70%, optionally at most 60% of a radius of the fluid transfer tube circumferentially adjacent to the reflux regions.

48. The fluid transfer tube according to claim 43, wherein one or both of:
a) the reflux regions are substantially longitudinal, said reflux regions being parallel to the longitudinal axis of the fluid transfer tube; and
b) the reflux regions extend along at least a portion of the fluid transfer tube that is configured to be in contact with the tissue when the distal end of the fluid transfer tube is inserted into the tissue.

49. The fluid transfer tube according to claim 43, wherein the reflux regions do not extend to the proximal end of the fluid transfer tube.

50. The fluid transfer tube according to claim 43, wherein the plurality of reflux regions comprises 5 reflux regions in total.

51. The fluid transfer tube according to claim 43, wherein the reflux regions comprise channels.

52. The fluid transfer tube according to claim 51, wherein an outer diameter of the fluid transfer tube and a depth of the channels permit the atraumatic insertion into the tissue.

53. The fluid transfer tube according to claim 51, wherein the channels have a radial depth of at least 0.05 mm, optionally at least 0.075 mm, optionally at least 0.1 mm.

54. The fluid transfer tube according to claim 43, wherein the outer surface of the fluid transfer tube in the reflux regions is concave and the outer surface of the fluid transfer tube circumferentially adjacent to the reflux regions is convex.

55. The fluid transfer tube according to claim 54, wherein the outer surface of the fluid transfer tube at the distal end is smoothly curved at all points around the circumference of the fluid transfer tube, and a maximum curvature of the outer surface of the fluid transfer tube circumferentially adjacent to the reflux regions is no greater than a maximum curvature of the outer surface of the fluid transfer tube in the reflux regions.

56. The fluid transfer tube according to claim 43, wherein a perimeter of the fluid transfer tube is at least 5% larger, optionally at least 10% larger, optionally at least 15% larger than an outer circumference of the fluid transfer tube.

57. The fluid transfer tube according to claim 43, wherein the outer surface of the fluid transfer tube is substantially flat in the reflux regions.

58. The fluid transfer tube according to claim 57, wherein the plurality of reflux regions comprises a total of 6 reflux regions and the distal end of the fluid transfer tube has a hexagonal profile.

59. The fluid transfer tube according to claim 43, wherein the fluid transfer tube is configured for insertion into the tissue through a guide tube comprising a proximal end and a distal end configured for insertion into tissue.

60. A neurosurgical apparatus comprising:
a guide tube comprising: a proximal end; and a distal end configured for insertion into tissue; and
the fluid transfer tube of claim 59.

61. The neurosurgical apparatus of claim 60, wherein the distal end of the fluid transfer tube is configured to project beyond a distal end of the guide tube into the tissue by a length that can be adjusted and/or fixed, andthe reflux regions do not extend along a portion of the fluid transfer tube that is configured to be within the guide tube when the distal end of the fluid transfer tube is inserted into the tissue.

62. The neurosurgical apparatus of claim 61, wherein the fluid transfer tube comprises a stop that engages with a proximal end of the guide tube and thereby sets the length by which the fluid transfer tube projects beyond the distal end of the guide tube.

63. The neurosurgical apparatus of claim 60, wherein a difference between an outer diameter of the fluid transfer tube and an outer diameter of the distal end of the guide tube is such as to form a step that limits reflux of the infusate proximal to the step when the fluid transfer tube is inserted into the tissue through the through-bore of the guide tube.

64. The fluid transfer tube of claim 43, wherein one or more of:
a) the tissue is animal tissue, optionally mammalian tissue, optionally human tissue;
b) the tissue is brain tissue, optionally brain parenchyma;
c) the infusate is a therapeutic fluid.
